(19) United States
(12) Patent Application Publication
(10) Pub. No.: US 2025/0205317 A1
(43) Pub. Date: Jun. 26, 2025

(54) METHODS AND COMPOSITIONS FOR THE TREATMENT OF STROKE

(71) Applicants: University of Maryland, Baltimore, Baltimore, MD (US); The United States Government as represented by the Department of Veterans Affairs, Washington, D.C., DC (US)

(72) Inventors: J. Marc Simard, Baltimore, MD (US); Vladimir Gerzanich, Elkridge, MD (US); Jesse Stokum, Catonsville, MD (US)

(21) Appl. No.: 18/852,356

(22) PCT Filed: Mar. 28, 2023
(86) PCT No.: PCT/US2023/065023

§ 371 Date: Sep. 27, 2024

Related U.S. Application Data
(60) Provisional application No. 63/324,492, filed on Mar. 28, 2022.

(51) Int. Cl. A61K 38/49; A61K 31/35; A61K 31/382; A61K 31/7034; A61K 31/7048; A61K 31/7056; A61P 25/00

(52) U.S. Cl. CPC A61K 38/49; A61K 31/35; A61K 31/382; A61K 31/7034; A61K 31/7048; A61K 31/7056; A61P 25/00; C12Y 304/21068

(57) ABSTRACT

The present invention provides methods for treating reperfusion edema in patients who have suffered a stroke, the methods comprising administering an effective amount of one or more inhibitors of sodium D-glucose cotransporter 2 (SGLT2). The invention further contemplates administration of SGLT2 inhibitors in combination with recombinant tissue plasminogen activator (rt-PA). Exemplary SGLT2 inhibitors include canagliflozin, dapagliflozin, empagliflozin, ertugliflozin, ipragliflozin, luseogliflozin, re-mogliflozin etabonate, sergliflozin etabonate, sotagliflozin, tofogliflozin and combinations thereof.

### CROSS-REFERENCE TO RELATED APPLICATIONS

This application claims the benefit of U.S. Provisional Appl. No. 63/324,492, filed on Mar. 28, 2022, the contents of which are hereby incorporated by reference in their entirety.

### FIELD OF THE INVENTION

The field of the invention generally relates to medicine and pharmaceuticals, in particular to methods of treating patients who have suffered from ischemic stroke.

### BACKGROUND OF THE INVENTION

Stroke is the second leading cause of death worldwide, and up to 50% survivors are left with chronic disability (Donkor E S, Stroke Res Treat, (2018), 3238165). In acute ischemic stroke (AIS), morbidity and mortality are determined by the amount and location of the tissues that are lost, as well as by secondary injury, the most important in the acute phase being brain swelling. The introduction of recombinant tissue plasminogen activator (rtPA) in 1995 led to substantial improvements in outcomes. National Institute of Neurological D and Stroke rt PASSG, N. Engl. J. Med. 1995, 333:1581-1587. More recently, mechanical thrombectomy has revolutionized the management of large vessel occlusion and has greatly improved outcomes. Powers, et al., Stroke 2019, 50: e344-e418. However, major challenges remain after reopening or recanalizing large cerebral arteries by either rtPA or mechanical thrombectomy, including salvaging neurons and mitigating reperfusion injury.

In non-lethal cerebral infarctions, brain swelling is an independent predictor of poor outcome, and in large hemispheric infarction (LHI), brain swelling places patients at high risk for neurological deterioration and is largely responsible for the high mortality rate of 50-80% (Battey et al., Stroke, (2014), 45:3643-3648; Arch et al., Curr Treat Options Cardiovasc Med, (2014), 16:275). The only proven treatment for severe brain swelling is decompressive craniectomy, a lifesaving but morbid surgical procedure that involves removing a large part of the cranium (Kurland et al., Neurocrit Care., (2015), 23:292-304).

Preclinical studies in rodent models of AIS have shown that the degree of brain swelling depends on infarct size (Kondo et al., J Neurosci, (1997), 17:4180-4189; Park et al., Acta Neurochir Suppl, 1997; 70:17-19). Moreover, most preclinical studies in rodents have shown that treatments that reduce swelling or edema do so only in proportion to the reduction in infarct volume conferred by the treatment. With few exceptions, no molecularly directed treatments have been put forth that dissociate brain swelling from infarct size (Woo et al., Neurosci Lett, (2020), 718:134729). However, effective clinical management of brain swelling requires molecularly informed treatments that act on infarcts of any size, without having to depend on a reduction in infarct volume. The strategy of reducing brain swelling independently of infarct size would be of great clinical significance, especially in LHI (Ng et al., Stroke, (2021), 52:3450-3458).

Following ischemia, brain swelling is linked to the aberrant or maladaptive function of sodium transporters at the blood-brain barrier (BBB) (Stokum et al., J Cereb Blood Flow Metab, (2016), 36:513-538; Song et al., Glia, (2020), 68:472-494; Stokum et al., Science Signaling, (2022), in review. Among the numerous sodium transporters known to be active, those belonging to the family of sodium-glucose co-transporters (SGLT) are understudied in brain ischemia (Pawlos et al., Molecules, (2021), 26). Several SGLT2 inhibitors of the gliflozin class are approved for the treatment of type 2 diabetes mellitus (T2DM) in humans, but the effect of these agents on brain swelling in preclinical models of AIS has not been evaluated (Abdel-Latif R G et al., Arch Pharm Res, (2020), 43:514-525; Amin et al., Fundam Clin Pharmacol, (2020), 34:548-558).

Reperfusion injury is characterized by loss of blood-brain barrier (BBB) integrity, which leads to edema formation (“reperfusion edema”), hemorrhagic conversion, brain swelling and neurological deterioration. Reperfusion injury limits the time window for thrombectomy, limits the volume of ischemic tissue that can be safely reperfused, (Mlynash, et al., Stroke 2011, 42:1270-1275; Inoue, et al., Stroke 2012, 43:2494-2496; Kimberly, et al., JAMA Neurol. 2018, 75:453-461) and limits neurological recovery with comorbidities such as hyperglycemia and others. Lindsberg, et al., Stroke 2004, 35:363-364; Mandava, et al., Transl. Stroke Res. 2014, 5:519-525. Treatments to reduce reperfusion injury would greatly expand the use of mechanical thrombectomy, would salvage neurons indirectly by preventing secondary injury, and would thereby further improve neurological outcomes.

Numerous mechanisms have been implicated in reperfusion injury, including oxidative stress, leukocyte infiltration, mitochondrial mechanisms, platelet activation and aggregation, complement activation and BBB disruption. Lin, et al., Biochem. Pharmacol. (Los Angel) 2016:5; Begum, et al., Glia 2018, 66:126-144. In broad terms, an abundance of preclinical work has shown that blockade or inhibition of these various mechanisms may be beneficial, but to date none has been translated into clinical practice.

Reperfusion edema is a critical component of reperfusion injury and is directly responsible for brain swelling. The classic teaching is that reperfusion edema is strongly coupled to infarct volume. In preclinical animal models, treatments shown to reduce or exacerbate edema typically reduce or exacerbate infarct volumes by a commensurate amount, and vice versa. Kondo, et al., J. Neurosci. 1997, 17:4180-4189; Kim, et al., J. Mol. Med. (Berl.) 2020, 98:875-886. No treatment has been shown to reduce formation of edema unless it also reduces infarct volume.

Unfortunately, tying edema reduction to infarct reduction cannot be translated into clinical practice. Neurologists treating stroke patients are confronted with an infarct as a fait accompli that cannot be reduced.

At present, the only treatment shown to decouple reperfusion edema from infarct volume is antisense oligodeoxynucleotide directed against 1) ATP-binding cassette transporter sub-family C member 8 (Abcc8), the gene that encodes sulfonylurea receptor-1 (SURI), which in turn regulates NC(Ca-ATP) nonselective cation channel, which mediates cerebral edema after ischemic stroke, or 2) transient receptor potential cation channel subfamily M member 4 (Trpm4), a gene that encodes a protein that serves as a calcium-activated nonselective cation channel, which mediates transport of monovalent cations across membranes. See Simard, et al., Nat. Med. 2006, 12 (4): 433-440; Simard, et al., J. Cereb. Blood Flow Metab. 2012, 32 (9): 1699-1717; Simard, Targeting NCCA-ATP channel for organ protection following ischemic episode, U.S. Patent Appl. Pub. No. 2019/0117672 A1; and U.S. Patent Appl. Pub. No. 2019/0218287 A1.

Astrocyte endfeet are known to play an important role in maintaining BBB integrity under normal and developmental conditions. Wolburg, et al., Cell Tissue Res. 2009, 335:75-96; Abbott, et al., Neurobiol. Dis. 2010, 37:13-25; Baeten, et al., Dev. Neurobiol. 2011, 71:1018-1039; Broux, et al., Semin. Immunopathol. 2015, 37:577-590. However, the function of astrocyte endfeet is poorly understood under conditions of post-ischemic reperfusion. Astrocyte endfoot swelling is a well-documented event post-ischemia, and the endfoot protein, aquaporin-4, is widely known to be important in edema formation. Zador, et al., Handb. Exp. Pharmacol. 2009:159-170; Berezowski, et al., Int. J. Cell Biol. 2012:176287; Jullienne, et al., Future Neurol. 2013, 8:677-689; Xiang, et al., Acta Neurochir. Suppl. 2016, 121:19-22; Verkman, et al., Expert Opin. Ther. Targets 2017, 21:1161-1170. However, the molecular machinery in astrocyte endfeet that governs reperfusion edema is unknown.

Glucose transporters (GLUT) exist in two major groups: (i) the GLUT energy-independent facilitated transporters and (ii) the sodium-D-glucose cotransporters (SGLT). Manolescu, et al., Physiology (Bethesda) 2007, 22:234-240. The SGLT are highly expressed in the kidney, where they are responsible for approximately 90% of renal glucose reabsorption in the proximal convoluted tubules. Wright, Am. J. Physiol. Renal Physiol. 2001, 280: F10-18; Wright, et al., Pflugers Arch. 2004, 447:510-518. By disabling SGLT2 activity with specific inhibitors of the gliflozin family (canagliflozin, dapagliflozin, empagliflozin, ertugliflozin, ipragliflozin, luseogliflozin, remogliflozin etabonate, sergliflozin etabonate, sotagliflozin, tofogliflozin), reabsorption of glucose into the bloodstream can be significantly diminished, leading to glycosuria and lowered serum glucose. Orally prescribed SGLT2 inhibitors are used as second-or third-line treatments for hyperglycemia in patients with diabetes mellitus type 2.

In the brain, glucose enters cells via the glucose transporters GLUT1-6, GLUT8 and SGLT1 and SGLT2. Koepsell, Pflugers Arch. 2020, 472:1299-1343. Whereas SGLT2 is expressed predominantly in kidney, it exhibits only minor expression in the brain. Chen, et al., Diabetes Ther. 2010, 1:57-92; Nishimura, et al., Drug Metab. Pharmacokinet. 2005, 20:452-477; Tazawa, et al., Life Sci. 2005, 76:1039-1050; Wright, et al., Physiol. Rev. 2011, 91:733-794. In the human brain, Sglt2 mRNA has been detected by RT-PCR, where it appears to be most strongly expressed in cerebellum. In a proteomic analysis, expression of Sglt2 mRNA was identified in microvessels isolated from rat brain cortex. Enerson, et al., J. Cereb. Blood Flow Metab. 2006, 26:959-973. SGLT also may be expressed by neurons. Erdogan, et al., BMC Neurol. 2018, 18:81. However, in a recent review on this topic (Koepsell, Pflugers Arch. 2020, 472:1299-1343), the physiological relevance of SGLT2/Sglt2 in brain has been questioned, since the expression of SGLT2/Sglt2 in brain is very low, and no data had been reported showing positive SGLT2/Sglt2-related signals by immunohistochemistry or in situ hybridization.

Given the prominence of stroke as a cause of mortality and disability in humans and the dearth of available therapeutic agents that can reduce edema after an infarction has occurred, there is great need in the art for new pharmaceuticals that can decouple edema from infarct volume, and that can be used as adjuvants, e.g., to the use of rtPA and mechanical thrombectomy.

The above information is presented solely for purposes of enhancing an understanding of the backdrop for the present invention and does not constitute any representation that it forms prior art or art that is generally known to those skilled in the field of the present invention.

### SUMMARY OF THE INVENTION

It is to be understood that both the present general description of the invention and the following detailed description are exemplary, and thus do not restrict the scope of the invention.

To date, few practical treatments have been advanced that shift the relationship between reperfusion edema and infarct volume. The concept of dissociating edema formation from infarct volume has great clinical significance. As described herein, shifting the relationship, or decoupling reperfusion edema from infarct volume can be used as an adjunctive therapy, for example for rtPA and mechanical thrombectomy in treating patients with stroke. Thrombectomy patients with an irreversible infarct would greatly benefit from therapies that ameliorate reperfusion edema independently of infarct severity.

In one aspect, the invention provides a method of treating reperfusion edema associated with ischemic stroke in a patient in need thereof, comprising administering an effective amount of one or more inhibitors of sodium D-glucose cotransporter 2 (SGLT2). In some embodiments, the inhibitor is a member of the gliflozin family of drugs. In some embodiments, the inhibitor is selected from the group consisting of canagliflozin, dapagliflozin, empagliflozin, ertugliflozin, ipragliflozin, luseogliflozin, remogliflozin etabonate, sergliflozin etabonate, sotagliflozin, tofogliflozin and combinations thereof.

In some embodiments, the SGLT2 inhibitor reduces reperfusion edema when administered after the patient has suffered an ischemic stroke.

In some embodiments, the administration of the SGLT2 inhibitor reduces reperfusion edema in a manner that is independent of infarct volume.

In some embodiments, the SGLT2 inhibitor is administered in combination with an effective amount of recombinant tissue plasminogen activator (rtPA).

In some embodiments, the SGLT2 inhibitor is administered in combination with mechanical thrombectomy.

In some embodiments, the treatment with the SGLT2 inhibitor results in an increased time window for thrombectomy.

In some embodiments, the treatment with the SGLT2 inhibitor does not significantly affect the serum glucose level of the patient.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. illustrates that reperfusion edema is strongly coupled to infarct volume. Diagram depicting the widely held understanding that reperfusion edema, here quantified as hemispheric swelling or enlargement, is directly related to infarct volume. The diagram is taken from FIG. 4A of Kondo et al., 1997 (Kondo, et al., referenced above), based on data from a mouse model of focal cerebral ischemia with 1-hour middle cerebral artery occlusion (MCAo) followed by 24 hours reperfusion; the empty vs. filled symbols represent controls vs. mice with reduced CuZn-superoxide dismutase activity.

FIG. 2. shows the present finding that SGLT2 is prominently expressed in perivascular endfeet. A,B: Immunolabeling of post-ischemic/post-reperfusion cortex for SGLT2 (green) and GFAP or β-dystroglycan (red) showing localization of SGLT2 in astrocyte endfeet (yellow).

FIG. 3. shows the decoupling edema from infarct volume. A,B: Edema, measured as percent hemispheric swelling, vs. TTC infarct volume following 2 hour MCAo and 24 hour reperfusion; data are shown for C57BL/6 mice treated upon reperfusion with vehicle (empty circles) vs. the SGLT2 inhibitor, canagliflozin (filled squares; 0.07 mg/kg IV). Note the significant downward shift in the regression line, signifying reduced coupling between reperfusion edema and infarct volume.

FIG. 4. shows that serum glucose was not affected in mice administered canagliflozin (0.07 mg/kg IV) vs. vehicle; 5 mice/group.

FIG. 5. SGLT2 is upregulated in astrocytes following MCAo/R. A-D: Merged images of double immunolabelings for SGLT2 (green) and neuronal NeuN (red; A,B) or astrocytic GFAP (red; C,D) in ipsilateral (Ipsi) and contralateral (Contra) cortex following MCAo/R ( 2/24 hours); inserts in (D) illustrate individual labelings and the merged image for one cell; co-localization appears as yellow/orange; nuclei stained with 4′,6-diamidino-2-phenylindole (DAPI); all bars, 25 μm; representative of data from 5 mice. E-G: RNAscope for Slc5a2 and Aquaporin4 (Aqp4) (E) with quantification (F,G) in ipsilateral (Ipsi) and contralateral (Contra) regions of interest, defined as 50-μm circles containing AQP4 loci and a DAPI-positive nucleus; 21 cells/group; **, P<0.01; 2-3 mice/group. H,I: Astrocytes were isolated from cortex of naïve mice, and ipsilateral and contralateral cortex of post-MCAo/R ( 2/6 hours), and were analyzed for Slc5a2 mRNA by qPCR (H) and for SGLT2 protein by immunoblot (I); cell isolations were from 3 mice for each condition; β-actin used as loading control; kidney tissue used as positive control (PC).

FIG. 6. Astrocyte swelling induced by a glycemic challenge is blocked by canagliflozin. A: tdTomato-expressing astrocyte in an ex vivo brain slice from an MCAo/R ( 2/6 hours) mouse at baseline (0 min) and 30 min after a glycemic challenge, induced by a step change in glucose from 2 to 10 mM; bar, 25 μm. B,C: Astrocyte volume changes in individual cells (B) and average changes (mean+SE) (C) in astrocytes from contralateral brain (Contra), ipsilateral brain (Ipsi), and ipsilateral brain treated with canagliflozin (Ipsi+CANA; 5 μM); **, P<0.01; 8 cells from 3 mice for each condition.

FIG. 7. Canagliflozin reduces brain swelling independently of infarct size. A: TTC-stained coronal sections following MCAo/R ( 2/24 hours) showing large infarcts with both vehicle (VEH) and canagliflozin (CANA; 200 μg/kg IV) administered at reperfusion, but reduced hemispheric swelling with CANA. B,C: Infarct volume (B) and hemispheric swelling (C) following MCAo/R ( 2/24 hours) in mice administered VEH or CANA at reperfusion; **, P<0.01; 43 or 25 mice/group. D: Ipsilateral and contralateral brain water following MCAo/R ( 2/24 hours) in mice administered VEH or CANA; *, P<0.05; 9 or 11 mice/group. E: Garcia scores following MCAo/R ( 2/24 hours) in mice administered VEH or CANA; same mice as in (B,C). F: Plot of hemispheric swelling vs. infarct volume; same data as in (B,C); data were fit to Eq. 1 in Methods; non-linear least squares fit gave values of k=0.030/mm³ vs. 0.036/mm³, and YM=17.9% vs. 33.2% (P<0.0001) for canagliflozin vs. vehicle, respectively.

### DETAILED DESCRIPTION OF THE INVENTION

In acute ischemic stroke, morbidity and mortality are greatly influenced by brain swelling. In preclinical studies, treatments that reduce swelling generally do so only in proportion to the reduction in infarct volume conferred by the treatment. However, effective clinical management of brain swelling requires molecularly informed treatments that act independently of a reduction in infarct volume. Sodium transporters have been implicated in post-ischemic brain swelling. The present disclosure examined the role of sodium glucose co-transporter 2 (SGLT2) on brain swelling.

The invention is based on the surprising discovery that sodium glucose cotransporter 2 (SGLT2) inhibitors, particularly the class of drugs known as gliflozins, e.g., canagliflozin, are effective in treating reperfusion edema in patients who have suffered a stroke.

The present inventors show herein that post-ischemic brain swelling is regulated by druggable cellular/molecular mechanisms such as SGLT2 that are distinct from those governing infarct size.

The present inventors have also shown that SGLT2 is localized in astrocyte endfeet, which are known to be involved in maintaining the integrity of the blood brain barrier (BBB). The BBB may be compromised in a patient having a stroke, where swelling of brain tissue may accompany or follow infarction, which is defined as obstruction of the blood supply that causes local death of tissue. The present inventors have further shown in mice that administration of canagliflozin, a member of the gliflozin family, reduced reperfusion edema relative to infarct volumes in comparison with the relationship between reperfusion edema and infarct volume in control mice that received no drug. This uncoupling of reperfusion edema from infarct volume represents a significant advance in stroke treatment.

Reference will now be made in detail to embodiments of the invention which, together with the drawings and the following examples, serve to explain the principles of the invention. These embodiments describe the invention in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

### Methods

In some embodiments, the invention provides a method of treating reperfusion edema associated with ischemic stroke in a patient in need thereof, comprising administering to the patient an effective amount of one or more inhibitors of sodium D-glucose cotransporter 2 (SGLT2).

In accordance with the claimed methods, the patient in need has suffered an ischemic stroke. The term “patient” refers to any animal (e.g., a mammal), including, but not limited to humans, non-human primates, rodents, and the like, which is to be the recipient of a particular treatment. Typically, the terms “subject” and “patient” are used interchangeably herein. In some embodiments, the patient is a human.

“Ischemia” is a condition in which the blood flow is restricted or reduced in a part of the body of a subject. As used herein, the term “stroke” refers to any acute, clinical event related to the impairment of cerebral circulation. The terms “acute cerebral ischemia” and “stroke” can be used interchangeably. A “stroke” occurs when the blood supply to any part of the brain of a subject is interrupted or reduced, preventing brain tissue from getting the oxygen and nutrients needed for normal function.

The terms “treating” and “treatment” as used herein refer to administering to a subject a therapeutically effective amount of a composition so that the subject has an improvement in the disease or condition. The improvement is any observable or measurable improvement. Thus, one of skill in the art realizes that a treatment may improve the patient's condition, but may not be a complete cure of the disease. Treating may also comprise treating subjects at risk of developing a disease and/or condition. As used herein, “treating” or “treatment” also encompasses a preventative treatment. The term “preventing” as used herein refers to minimizing, reducing or suppressing the risk of developing a disease state or parameters relating to the disease state or progression of other abnormal or deleterious conditions.

As used herein, the terms “effective amount” or “therapeutically effective amount” are interchangeable and refer to an amount that results in an improvement or remediation of at least one symptom of the disease or condition. Those of skill in the art understand that the effective amount may improve the patient's or subject's condition, but may not be a complete cure of the disease and/or condition.

In some embodiments, the SGLT inhibitor(s) can reduce reperfusion edema when administered after the patient has suffered from ischemic stroke. “Edema” in a particular body tissue is swelling of that tissue caused by excess fluid trapped there. “Reperfusion edema” refers to the swelling of tissue caused by excess fluid caused when blood supply returns to the tissue after a period of ischemia or oxygen deprivation. “Reperfusion injury,” also called ischemia-reperfusion injury or reoxygenation injury, refers to tissue damage caused when blood supply returns to tissue after a period of ischemia or oxygen deprivation. Reperfusion injury is characterized by loss of blood-brain barrier (BBB) integrity, which leads to edema formation (“reperfusion edema”), hemorrhagic conversion, brain swelling and neurological deterioration. As used herein, the terms “blood brain barrier” or “BBB” refer the barrier between brain blood vessels and brain tissues whose effect is to restrict what may pass from the blood into the brain.

In certain embodiments, administration of the SGLT inhibitor(s) can reduce reperfusion edema in a manner that is independent of infarct volume. “Infarct volume” refers to the volume of necrosis (death) in a tissue or organ resulting from obstruction of the local blood circulation. Necrosis cannot be reversed.

The SGLT2 inhibitor that can be used in the methods of the invention is not necessarily limiting. In some embodiments, the administration of the SGLT2 inhibitor does not significantly affect the serum glucose level of the patient.

As used herein, the term “inhibit” refers to the ability of the compound to block, partially block, interfere, decrease, reduce or deactivate a receptor such as SGLT2. Thus, one of skill in the art understands that the term “inhibit” encompasses a complete and/or partial loss of activity of the receptor. Receptor activity may be inhibited by blockage of ligand binding sites on the receptor, by interference with the mechanism of expression of the receptor protein, or by other means. In some embodiments, expression of SGLT2 is inhibited, for example, by one or more nucleic acids. In some embodiments, the inhibitor acts directly on the receptor.

In some embodiments, the inhibitor is a member of the gliflozin family of drugs. Gliflozins are inhibitors of the sodium D-glucose cotransporter SGLT2, a protein that is involved in reabsorption of glucose in the kidney. Gliflozins as a class have in common a glucose moiety that has an aromatic group attached covalently at the beta position of anomeric carbon 1, where the aromatic group has a diarylmethylene structure. The gliflozins can be prepared using methods that are generally known in organic chemistry. Larson, The synthesis of gliflozins, Chimica Oggi-Chemistry Today 2015, 33 (2): 37-40. Some of the gliflozins are available commercially. For example, danagliflozin is available from Sigma-Aldrich, St. Louis, MO. Empagliflozin is also available from Sigma-Aldrich, sourced from chemical wholesaler Ambeed, Inc., Arlington Heights, IL. Gliflozins have been used to decrease blood sugar levels, e.g., in the treatment of type 2 diabetes mellitus.

In some embodiments, the one or more inhibitors can be selected from the group consisting of canagliflozin, dapagliflozin, empagliflozin, ertugliflozin, ipragliflozin, luseogliflozin, remogliflozin etabonate, sergliflozin etabonate, sotagliflozin, tofogliflozin and combinations thereof.

In some embodiments, the SGLT2 inhibitor can be any one of the compounds described in U.S. Pat. Nos. 10,836,753; 10,815,210; 10,696,662; 10,555,958; 10,544,135; 10,533,032; 9,834,533; 9,757,404; 9,725,478; 9,573,959; 9,453,039; 9,371,303; 9,340,521; 9,198,925; 9,174,971; 9,006,403; 9,006,187; 8,999,941; 8,921,412; 8,883,743; 8,791,077; 8,716,251; 8,685,934; 8,603,989; 8,586,550; 8,541,380; 8,518,895; 8,501,698; 8,450,286; 8,362,232; 8,283,454; 8,153,649; 8,088,743; 8,084,436; 7,919,598; 7,851,502; 7,375,213; 6,936,590; 6,555,519; and 6,414,126, which compounds and their methods of making are herein incorporated by reference in their entirety. In some embodiments, the SGLT2 inhibitor can be any one of the compounds described in WO 2011/070592, which compounds and their methods of making are herein incorporated by reference in their entirety.

The SGLT2 inhibitors, such as the gliflozin drugs, can be administered according to the present invention in a variety of ways, and the mode of administration is not particularly limiting. In some embodiments, the agent is administered topically, intravenously, subcutaneously, transcutaneously, intrathecally, intraventricularly, intramuscularly, intracutaneously, intragastrically, transnasally, or orally. In some embodiments, a daily dosage of inhibitor, such as a gliflozin drug, is administered orally, for example, as a plurality of dosages administered at intervals over a portion of the day or throughout the day.

In some embodiments, an effective amount of SGLT2 inhibitor, such as a gliflozin drug, that is administered daily includes a dose of from about 5 mg to about 300 mg. In some embodiments, an amount of SGLT2 inhibitor, such as a gliflozin drug, administered is from about 5 mg to about 10 mg; about 10 mg to about 15 mg; about 15 mg to about 25 mg; about 25 mg to about 35 mg; about 35 mg to about 50 mg; about 50 mg to about 65 mg; about 65 mg to about 85 mg; about 85 mg to about 105 mg; about 105 mg to about 125 mg; about 125 mg to about 150 mg; about 150 mg to about 175 mg; about 175 mg to about 200 mg; about 200 mg to about 225 mg; about 225 mg to about 250 mg; about 250 mg to about 275 mg; and about 275 mg to about 300 mg. Of course, all of these amounts are exemplary, and any amount in-between these points is also expected to be of use in the invention.

In some embodiments, an effective amount of SGLT2 inhibitor, such as a gliflozin drug, that is administered daily includes a dose of about 0.0001 μg/kg/day to about 20 mg/kg/day. In some embodiments, an effective amount of SGLT2 inhibitor, such as a gliflozin drug, that is administered daily includes a dose of about 1.0 μg/kg/day to about 5 mg/kg/day. In some embodiments, an effective amount of SGLT2 inhibitor, such as a gliflozin drug, that is administered daily includes a dose of about 0.02 mg/kg body weight of the subject to about 3 mg/kg body weight of the subject. In some embodiments, an amount of SGLT2 inhibitor, such as a gliflozin drug, administered is from about 0.02 mg/kg to about 0.05 mg/kg; about 0.05 mg/kg to about 0.1 mg/kg; about 0.1 mg/kg to about 0.15 mg/kg; about 0.15 mg/kg to about 0.25 mg/kg; about 0.25 mg/kg to about 0.4 mg/kg; about 0.4 mg/kg to about 0.6 mg/kg; about 0.6 mg/kg to about 0.9 mg/kg; about 0.9 mg/kg to about 1.3 mg/kg; about 1.3 mg/kg to about 1.8 mg/kg; about 1.8 mg/kg to about 2.4 mg/kg; and about 2.4 mg/kg to about 3.0 mg/kg. Of course, all of these amounts are exemplary, and any amount in-between these points is also expected to be of use in the invention.

In some embodiments, the total daily dose of the active compounds of the present invention administered to a subject in single or in divided doses. Single dose compositions may contain such amounts or submultiples thereof to make up the daily dose.

One of skill in the art will understand that exact dosages can depend on the subject patient's health history and/or previous responses to the subject compound(s) or composition(s) and will be ascertainable by a person skilled in the art using known methods and techniques for determining effective doses.

As provided herein, the subject can be administered a single dose or multiple doses of SGLT2 inhibitor, such as a gliflozin drug, or a plurality of inhibitors. The dose of the drugs, e.g., a plurality of gliflozin drugs, can be administered in a single composition or in multiple compositions. Thus, the administration includes co-administration, using separate formulations or a single pharmaceutical formulation, and consecutive administration in any order, wherein preferably there is a time period while each active agent simultaneously exerts its biological activities. In some embodiments, a plurality of gliflozin drugs is administered in a single composition. In some embodiments, the single composition of either a single gliflozin drug or a plurality of gliflozin drugs comprises an orally administered slow or delayed release tablet or capsule.

In some embodiments, the inhibitor can be administered parenterally or alimentarily. Parenteral administrations include, but are not limited to, intravenously, intradermally, transdermally, intramuscularly, intraarterially, intrathecally, subcutaneously, or intraperitoneally. See, e.g., U.S. Pat. Nos. 6,613,308, 5,466,468, 5,543,158; 5,641,515; and 5,399,363 (each specifically incorporated herein by reference in its entirety). Alimentary administrations include, but are not limited to orally, buccally, rectally, or sublingually.

In some embodiments, the administration of the therapeutic compounds of the present invention may include systemic, local and/or regional administrations, for example, topically (dermally, transdermally), via catheters, implantable pumps, dermal patches, transdermal patches, etc. Other routes of administration are also contemplated such as, for example, arterial perfusion, intracavitary, intraperitoneal, intrapleural, intraventricular and/or intrathecal. The skilled artisan is capable of determining the appropriate administration route using standard methods and procedures. Other routes of administration are discussed elsewhere in the specification and are incorporated herein by reference.

Treatment methods involve treating an individual with an effective amount of a composition comprising one or more SGLT2 inhibitors, such as gliflozin drugs. An effective amount is described, generally, as that amount sufficient to detectably and repeatedly ameliorate, reduce, minimize, or limit the extent of a deleterious condition of a patient. The effective amount of one or more SGLT2 inhibitors to be used includes those amounts effective to produce beneficial results, particularly with respect to amelioration of reperfusion edema resulting from stroke in the recipient patient.

As is well known in the art, a specific dose level of active compounds such as the one or more gliflozin drugs for any particular patient depends upon a variety of factors including the activity of the specific compound employed, the age, body weight, general health, sex, diet, time of administration, route of administration, rate of excretion, drug combination, and the severity of the particular disease or condition undergoing therapy.

In some embodiments, the SGLT2 inhibitor can be administered alone or in combination with one or more active pharmaceutical agents or treatments. The types of active agents or treatments that can be administered with the SGLT2 inhibitors are not limiting.

In some embodiments, the SGLT2 inhibitor is administered with one or more additional active pharmaceutical agents or treatments that are useful to treat stroke or any of its effects in the subject. In some embodiments, the inhibitor can be used as an adjunct to treatments for stroke, such as recombinant tissue plasminogen activator (rtPA) or mechanical thrombectomy to reduce or prevent brain edema regardless of infarct size.

The combined administration includes co-administration, using separate formulations or a single pharmaceutical formulation, and consecutive administration in either order, wherein in some embodiments, there is a time period while both (or all) active agents simultaneously exert their biological activities.

In some embodiments, the SGLT2 inhibitor is administered in combination with a surgical procedure, such as thrombectomy.

In some embodiments, the SGLT inhibitor(s) is/are administered in combination with mechanical thrombectomy. In some embodiments, the SGLT2 inhibitor is administered in combination with the use of a mechanical thrombolytic device (e.g. the Concentric MERCI device).

In some embodiments, the treatment with SGLT2 inhibitor can result in an increased time window for performing thrombectomy.

The time window can vary between patients. However, in some embodiments, even after several hours, mechanical thrombectomy may be indicated when magnetic resonance imaging (MRI) shows a relatively small area of acute infarction, and suggests a large ischemic penumbra confirmed with cerebral perfusion analysis. In such cases, the outcome with mechanical thrombectomy can be improved by treatment with a SGLT2 inhibitor such as gliflozin.

In some embodiments, the SGLT2 inhibitor is administered in combination with an effective amount of a therapeutic agent.

In some embodiments, the SGLT2 inhibitor is administered in combination with one or more other therapies or treatments.

In some embodiments, the one or more other therapies or treatments include mannitol, hypertonic saline or other hyperosmolar agent, decompressive craniectomy, or any standard therapy for brain edema and brain swelling.

In some embodiments, the SGLT2 inhibitor is administered in combination with an effective amount of a thrombolytic agent (e.g., tissue plasminogen activator (tPA), urokinase, prourokinase, streptokinase, anistreplase, reteplase, or tenecteplase).

In some embodiments, the SGLT2 inhibitor is administered in combination with an effective amount of an anticoagulant or antiplatelet (e.g., aspirin, warfarin or coumadin), statin, diuretic, vasodilator (e.g., nitroglycerin), mannitol, diazoxide or similar compounds that stimulate or promote an ischemic precondition.

In some embodiments, the SGLT2 inhibitor is administered in combination with an effective amount of recombinant tissue plasminogen activator (rtPA).

In some embodiments, the SGLT2 inhibitor is administered in combination with a) a SUR1 antagonist; or b) a Transient Receptor Potential cation channel subfamily M member 4 (TRPM4) antagonist. The Sur1-Trpm4 channel is a NC_{Ca-ATP} channel. As used herein, the term “NC_{Ca-ATP} channel” refers to a non-selective cation channel complex that is activated by intracellular calcium and blocked by intracellular ATP, and has a single-channel conductance to potassium ion (K+) of between about 20 and about 50 pS at physiological potassium concentrations. This channel complex includes a SURI receptor and is sensitive to SURI agonists and antagonists. In certain embodiments, the channel complex includes a pore that has similar properties to the TRPM4 channels, including blockade by TRPM4 blockers (such as, e.g., flufenamic acid, mefanimic acid, and niflumic acid), and therefore the pore of the NCC_{a-ATP} channel complex is TRPM4 channel. This channel complex is referred to herein as a “channel” and is described in greater detail elsewhere in the application. As used herein, the term “TRPM4 channel” refers to a pore that passed ions that is a member of the transient receptor potential channel family (hence the acronym “TRP”) and is the pore forming portion of the SUR1-sensitive NCC_{a-ATP} channel. The NCC_{a-ATP} channel was identified first in native reactive astrocytes (NRAs) and later in neurons and capillary endothelial cells after stroke or traumatic brain or spinal cord injury (see at least International Application WO 03/079987 which is incorporated by reference herein in its entirety). As with the KATP channel in pancreatic cells, the NCC_{a-ATP} channel is considered to be a heteromultimer structure comprised of sulfonylurea receptor type 1 (SUR1) regulatory subunits and pore-forming subunits. The pore-forming subunits have been characterized biophysically and have been identified as TRPM4. Examples of such compounds include an inhibitor of the channel, such as, for example, an antagonist of a type 1 sulfonylurea receptor, such as sulfonylureas like glibenclamide and tolbutamide, as well as other insulin secretagogues such as repaglinide, nateglinide, meglitinide, mitiglinide, iptakalim, endosulfines, LY397364, LY389382, gliclazide, glimepiride, MgADP, and combinations thereof. Other such channel inhibitors for use in the methods of the invention are TRPM4 inhibitors, for example, flufenamic acid, mefanimic acid, and niflumic acid.

### Compositions

The present invention also contemplates therapeutic methods employing compositions comprising the active substances disclosed herein, including compositions comprising combinations of active agents. In some embodiments, these compositions include pharmaceutical compositions comprising a therapeutically effective amount of one or more of the active compounds or substances along with a pharmaceutically acceptable carrier.

In another embodiment, the invention provides a pharmaceutical composition comprising an effective amount of a SGLT2 inhibitor, e.g., as described herein, and an additional active agent. In some embodiments, the additional active agent is useful to treat stroke or any of its effects in the subject. In some embodiments, the composition comprises an effective amount of an SGLT2 inhibitor and one or more of an effective amount of the following: a thrombolytic agent (e.g., tissue plasminogen activator (tPA), urokinase, prourokinase, streptokinase, anistreplase, reteplase, tenecteplase), an anticoagulant or antiplatelet (e.g., aspirin, warfarin or coumadin), statins, diuretics, vasodilators, mannitol, diazoxide or similar compounds that stimulate or promote ischemic precondition or a pharmaceutically acceptable salt thereof. In some embodiments, the inhibitor comprises a gliflozin compound that inhibits SGLT2 or a pharmaceutically acceptable salt thereof. This pharmaceutical composition can be considered neuroprotective, in some embodiments.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, alpha-tocopherol and the like; and the metal chelating agents such as citric acid, ethylenediamine tetraacetic acid (EDTA), sorbitol, tartaric acid, phosphoric acid and the like.

The active agents of the present invention can be administered alone or in combination with one or more active pharmaceutical agents. Liquid dosage forms for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring and perfuming agents.

Suppositories for rectal administration of the drug can be prepared by mixing the drug with a suitable non-irritating excipient, such as cocoa butter and polyethylene glycol which are solid at ordinary temperature but liquid at the rectal temperature and will, therefore, melt in the rectum and release the drug.

Solid dosage forms for oral administration may include capsules, tablets, pills, powders, gelcaps and granules. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, capsules, pills, and granules can be prepared with coatings and shells such as enteric coatings and other release-controlling coatings well known in the pharmaceutical formulating art. They may optionally contain opacifying agents and can also be of a composition that they release the active ingredient(s) only, or preferably, in a certain part of the intestinal tract, optionally in a delayed manner. Examples of embedding compositions which can be used include polymeric substances and waxes.

Dosage forms for topical or transdermal administration of a compound of this invention further include ointments, pastes, creams, lotions, gels, powders, solutions, sprays, inhalants or patches. Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In one embodiment, the therapeutic compound is delivered transdermally. The term “transdermal delivery” as used herein means administration of the pharmaceutical composition topically to the skin wherein the active ingredient or its pharmaceutically acceptable salts, will be percutaneously delivered in a therapeutically effective amount.

In some embodiments, the composition to be applied transdermally further comprises an absorption enhancer. The term “absorption enhancer” as used herein means a compound which enhances the percutaneous absorption of drugs. These substances are sometimes also referred to as skin-penetration enhancers, accelerants, adjuvants and sorption promoters. Various absorption enhancers are known to be useful in transdermal drug delivery. U.S. Pat. Nos. 5,230,897, 4,863,970, 4,722,941, and 4,931,283 disclose some representative absorption enhancers used in transdermal compositions and for topical administration. In some embodiments, the absorption enhancer is N-lauroyl sarcosine, sodium octyl sulfate, methyl laurate, isopropyl myristate, oleic acid, glyceryl oleate or sodium lauryl sulfoacetate, or a combination thereof. In some embodiments, the composition contains on a weight/volume (w/v) basis the absorption enhancer in an amount of about 1-20%, 1-15%, 1-10% or 1-5%. In some embodiments, to enhance further the ability of the therapeutic agent(s) to penetrate the skin or mucosa, the composition can also contain a surfactant, an azone-like compound, an alcohol, a fatty acid or ester, or an aliphatic thiol.

In one embodiment, the therapeutic compound is delivered via a transdermal patch. In some embodiments, the transdermal composition can further comprise one or more additional excipients. Suitable excipients include without limitation solubilizers (e.g., C₂-C₈ alcohols), moisturizers or humectants (e.g., glycerol [glycerin], propylene glycol, amino acids and derivatives thereof, polyamino acids and derivatives thereof, and pyrrolidone carboxylic acids and salts and derivatives thereof), surfactants (e.g., sodium lauryl sulfate and sorbitan monolaurate), emulsifiers (e.g., cetyl alcohol and stearyl alcohol), thickeners (e.g., methyl cellulose, ethyl cellulose, hydroxymethyl cellulose, hydroxypropyl cellulose, polyvinylpyrrolidone, polyvinyl alcohol and acrylic polymers), and formulation bases or carriers (e.g., polyethylene glycol as an ointment base). As a non-limiting example, the base or carrier of the composition can contain ethanol, propylene glycol and polyethylene glycol (e.g., PEG 300), and optionally an aqueous liquid (e.g., isotonic phosphate-buffered saline).

In some embodiments, the compound(s) or composition(s) can be administered to the subject once, such as by a single injection or deposition at or near the site of interest. In some embodiments, the compound(s) or composition(s) can be administered to a subject over a period of days, weeks, months or even years. In some embodiments, the compound(s) or composition(s) is administered at least once a day to a subject. Where a dosage regimen comprises multiple administrations, it is understood that the effective amount of the compound(s) or composition(s) administered to the subject can comprise the total amount of the compound(s) or composition(s) administered over the entire dosage regimen.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid can be used in the preparation of injectables.

The injectable formulation can be sterilized, for example, by filtration through a bacteria-retaining filter, or by incorporating sterilizing agents in the form of sterile solid compositions, which can be dissolved or dispersed in sterile water or other sterile injectable medium just prior to use.

In order to prolong the effect of a drug, it can be desirable to slow the absorption of a drug from subcutaneous or intramuscular injection. The most common way to accomplish this is to inject a suspension of crystalline or amorphous material with poor water solubility. The rate of absorption of the drug becomes dependent on the rate of dissolution of the drug, which is, in turn, dependent on the physical state of the drug, for example, the crystal size and the crystalline form. Another approach to delaying absorption of a drug is to administer the drug as a solution or suspension in oil. Injectable depot forms can also be made by forming microcapsule matrices of drugs and biodegradable polymers, such as polylactide-polyglycoside. Depending on the ratio of drug to polymer and the composition of the polymer, the rate of drug release can be controlled. Examples of other biodegradable polymers include polyorthoesters and polyanhydrides. The depot injectables can also be made by entrapping the drug in liposomes or microemulsions, which are compatible with body tissues.

As employed herein, the phrase “suitable dosage levels” refers to levels of inhibitor compound such as a gliflozin compound(s) sufficient to provide circulating concentrations high enough to effectively block SGLT2 and prevent or reduce reperfusion edema in vivo.

In accordance with a particular embodiment of the present invention, compositions comprising at least one or more gliflozin drugs and a pharmaceutically acceptable carrier are contemplated.

Exemplary pharmaceutically acceptable carriers include carriers suitable for oral, intravenous, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use. The active compound is admixed under sterile conditions with a pharmaceutically acceptable carrier and any needed preservatives or buffers as may be required.

The treatments may include various “unit doses.” Unit dose is defined as containing a predetermined quantity of the therapeutic composition (one or more gliflozin drugs) calculated to produce the desired responses in association with its administration, e.g., the appropriate route and treatment regimen. The quantity to be administered, and the particular route and formulation, are within the skill of those in the clinical arts. Also of importance is the subject to be treated, in particular, the state of the subject and the protection desired. A unit dose need not be administered as a single injection but may comprise continuous infusion over a set period of time.

In some embodiments, pharmaceutical compositions of the present invention comprise effective amounts of one or more gliflozin drugs (and, optionally, additional agent(s)) dissolved or dispersed in a pharmaceutically acceptable carrier. The preparation of a pharmaceutical composition that contains at least one inhibitor of SGLT2, e.g., a gliflozin drug, optionally, one or more additional active ingredient(s) will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity and general safety and purity standards as required by the FDA Office of Biological Standards.

The compositions can comprise different types of carriers depending on whether it is to be administered in solid, liquid or aerosol form, and whether it needs to be sterile for such routes of administration as injection. The present invention can be administered intravenously, intradermally, transdermally, intrathecally, intraventricularly, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art. In a specific embodiment of the present invention, the composition is combined or mixed thoroughly with a semi-solid or solid carrier. The mixing can be carried out in any convenient manner such as grinding.

In further embodiments, the present invention may concern the use of a pharmaceutical lipid vehicle composition that includes a combination of one or more gliflozin drugs, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the one or more gliflozin drugs may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic and/or prophylactic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Pharmaceutical formulations may be administered by any suitable route or means, including alimentary, parenteral, topical, mucosal or other route or means of administration. Alimentary routes of administration include administration oral, buccal, rectal and sublingual routes. Parenteral routes of administration include administration include injection into the brain parenchyma, and intravenous, intradermal, intramuscular, intraarterial, intrathecal, subcutaneous, intraperitoneal, and intraventricular routes of administration. Topical routes of administration include transdermal administration.

In some embodiments of the present invention, the one or more gliflozin drugs are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier or they may be enclosed in hard-or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (see, e.g., U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup or elixir may contain the active compound sucrose as a sweetening agent, methyl and propylparabens as preservatives, a dye and/or flavoring, such as cherry or orange flavor. Of course, any material used in preparing any dosage unit form should be pharmaceutically pure and substantially non-toxic in the amounts employed. In addition, the active compounds may be incorporated into sustained-release preparations and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In further embodiments, the one or more SGLT2 inhibitors such as gliflozin drugs may be administered via a parenteral route. As used herein, the term “parenteral” includes routes that bypass the alimentary tract. Specifically, the pharmaceutical compositions disclosed herein may be administered for example, but not limited to intravenously, intradermally, transdermally, intramuscularly, intraarterially, intraventricularly, intrathecally, subcutaneous, or intraperitoneally. See, e.g., U.S. Pat. Nos. 6,7537,514; 6,613,308; 5,466,468; 5,543,158; 5,641,515; and 5,399,363 (each specifically incorporated herein by reference in its entirety).

In some embodiments, the therapeutic compound is administered intrathecally. In some embodiments, the compound is administered intrathecally via an implantable pump. In one embodiment, the implantable pump comprises a SynchroMed™ II pump that stores and delivers medication into the intrathecal space (Medtronic).

Solutions of the active compounds or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, dimethyl sulfoxide (DMSO), polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In other embodiments, the one or more gliflozin drugs may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin.

Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch.” For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described, e.g., in U.S. Pat. Nos. 5,756,353 and 5,804,212 (each specifically incorporated herein by reference in its entirety). Likewise, the delivery of drugs using intranasal microparticle resins and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid or liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

All publications herein are incorporated by reference to the same extent as if each individual publication or patent application was specifically and individually indicated to be incorporated by reference.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Examples

### Example 1: SGLT2 in Astrocyte Endfeet of Microvessels

SGLT2 expression was studied in the brains of mice following 2-hour middle cerebral artery occlusion (MCAo) and 24 hours reperfusion. SGLT2 was found to be prominently expressed in elongated structures consistent with microvessels (FIG. 2). This finding is in accord with the aforementioned report based on proteomic analysis, in which Sglt2 mRNA was identified in microvessels isolated from rat brain cortex. Enerson, et al. (referenced above).

Brain microvessels are comprised of several cellular constituents, including a continuous tubular layer of endothelial cells joined by tight junctions, surrounded by a gliovascular basement membrane, within which reside pericytes and outside of which is an ensheathing layer of astrocyte endfoot processes. Co-immunolabeling experiments were performed to determine which of these cellular elements-endothelium, pericytes or astrocyte endfeet-expressed SGLT2. Co-immunolabeling for SGLT2 and glial fibrillary acidic protein (GFAP) or for SGLT2 and β-dystroglycan showed clear localization of SGLT2 in astrocyte endfeet, not endothelium or pericytes (FIG. 2).

### Example 2: SGLT2 Inhibition and Reperfusion Edema

The known variability in the brain damage caused by ischemia in C57BL/6 mice was leveraged (Knauss, et al., Frontiers in Neuroscience 2020) to quantify the relationship between reperfusion edema and infarct volume, under both control conditions and with SGLT2 inhibition. Mice underwent 2-hour MCAo followed by 24 hours reperfusion. At 2 hours, at the time of reperfusion, mice were randomly assigned to receive either vehicle control or canagliflozin (0.07 mg/kg IV). At 24 hours, the brains were evaluated for infarct volume using 2,3,5-triphenyltetrazolium chloride (TTC) (Khan, et al., Evaluation of an optimal temperature for brain storage in delayed 2, 3,5-triphenyltetrazolium chloride staining, J. Neurosci. Methods 2000, 98:43-47), and the hemisphere volume was quantified as a measure of reperfusion edema. McBride, et al., Correcting for Brain Swelling's Effects on Infarct Volume Calculation After Middle Cerebral Artery Occlusion in Rats, Transl. Stroke Res. 2015, 6:323-338. Infarct volumes were large, in accord with the severe ischemic insult, with no effect of canagliflozin on infarct volumes compared to vehicle controls (63.1±8.0 vs. 60.3±8.7% of hemisphere; p=0.33).

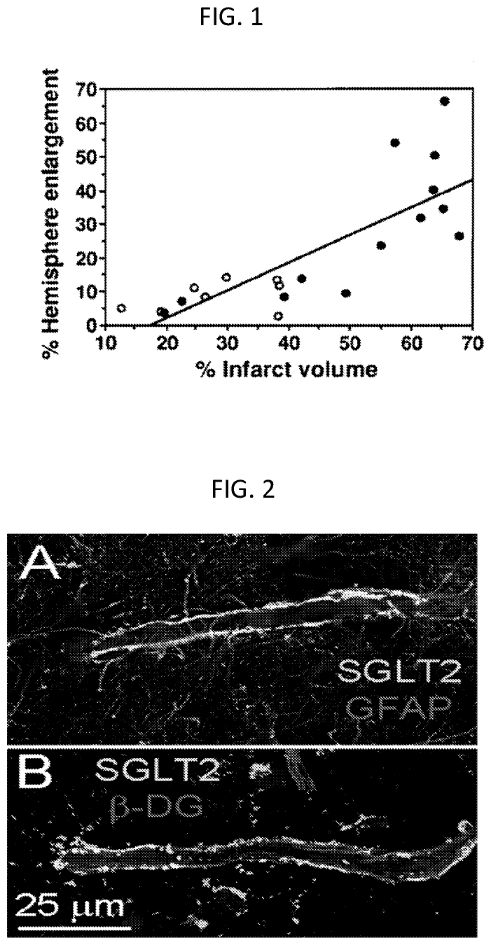

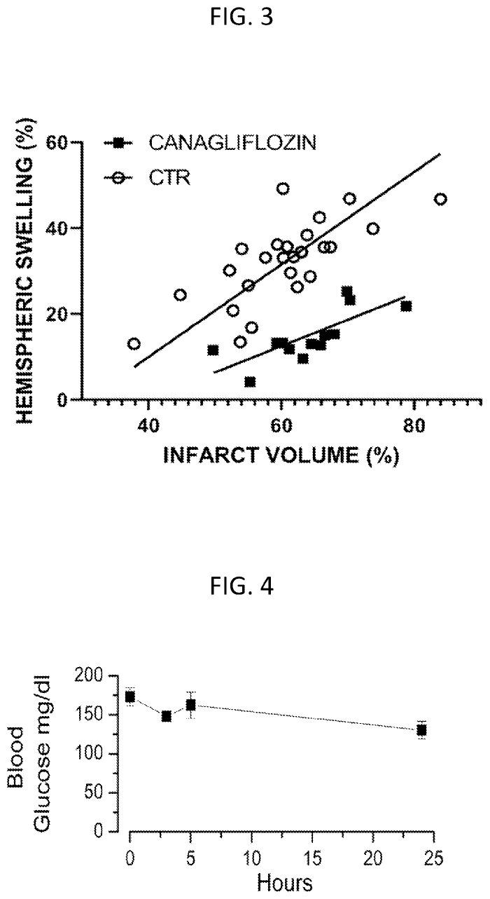

In the control mice, reperfusion edema was strongly related to infarct volume (Pearson's r²=0.53; p<0.01; FIG. 3), as reported (see FIG. 1). In mice administered canagliflozin at the onset of reperfusion, reperfusion edema was reduced at all infarct volumes (shift in Deming regression; p<0.0001; FIG. 3). The significant downward shift in the regression line with canagliflozin indicated a major reduction in coupling between reperfusion edema and infarct volume.

### Example 3: Effect of canagliflozin on serum glucose

Drugs of the gliflozin class are used in patients with diabetes mellitus to lower serum glucose. To control for the possibility that the effect of canagliflozin was mediated by a reduction in serum glucose, we measured serum glucose at various times before and after canagliflozin (0.07 mg/kg IV) administration. Compared to baseline, the mice remained euglycemic within the entire timeframe during which reperfusion edema was measured (FIG. 4).

Together, these data point to SGLT2 in the astrocyte endfoot as a primary determinant of BBB integrity during post-ischemic reperfusion, and indicate that SGLT2 plays a key role in coupling reperfusion edema to infarct volume.

While the invention has been described in connection with specific and preferred embodiments thereof, it is capable of further modifications without departing from the spirit and scope of the invention. This application is intended to cover all variations, uses, or adaptations of the invention, following, in general, the principles of the invention and including such departures from the present disclosure as come within known or customary practice within the art to which the invention pertains, or as are obvious to persons skilled in the art, at the time the departure is made. It should be appreciated that the scope of this invention is not limited to the detailed description of the invention hereinabove, which is intended merely to be illustrative, but rather comprehends the subject matter defined by the following claims.

### Example 4: Canagliflozin, an Inhibitor of Sodium/Glucose Co-Transporter 2, Reduces Ischemic Brain Swelling Independently of Infarct Size

The present example demonstrates that post-ischemic brain swelling is regulated by druggable cellular/molecular mechanisms such as SGLT2 that are distinct from those governing infarct size.

It is shown that Slc5a2/SGLT2 mRNA and protein were upregulated de novo in astrocytes following MCAo/R. In ex vivo MCAo/R brain slices, SGLT activation caused an increase in astrocyte cell volume that was blocked by canagliflozin. In MCAo/R mice, infarct volumes were 14-151 mm³ with no difference between vehicle and canagliflozin groups. The relationship between ipsilateral hemispheric volume and infarct volume was well described by a simple exponential function with maximum swelling of 17.9% vs. 33.2% (P<0.0001) for canagliflozin vs. vehicle, respectively, consistent with a reduction in swelling independent of infarct volume. Canagliflozin yielded better neurological function, reflecting the benefit of reduced swelling.

In the present example, mice underwent middle cerebral artery occlusion (2 hours) followed by 24-hours reperfusion (MCAo/R). At reperfusion, mice were administered vehicle vs. the SGLT2 inhibitor, canagliflozin. Infarct volume, hemispheric volume, and neurological function were measured using unbiased methods. Brain tissues were studied for expression of Slc5a2/SGLT2 and swelling of astrocytes.

### Methods

Subjects. C57BL6 mice were obtained from Envigo (Indianapolis, IN). All experiments were carried out using male mice (22-30 gm); 105 mice underwent MCAo/R (2-hour occlusion) followed by reperfusion and were euthanized at 6 hours (6 mice) or 24 hours (99 mice). Three uninjured mice were used as controls for astrocyte isolation.
MCAo/R was induced as described using a silicon filament occluder (602356PK5Re Doccol Corp) and common carotid artery ligation. Mice were excluded if: (1) they did not exhibit circling behavior before reperfusion; (2) they died before the time of planned euthanasia (24 hours); (3) subarachnoid hemorrhage was diagnosed at necropsy (Bertrand et al., J Vis Exp, (2017)). No animal was excluded based on infarct size.
Treatment. At the time of reperfusion, the external jugular vein was exposed, and mice were randomly assigned to receive vehicle or canagliflozin (200 μg/kg) intravenously (IV). The scientist performing the surgery and drug administration was “blinded” to treatment group.
Blood glucose was measured at reperfusion and at euthanasia using a glucometer.
Neurological function was evaluated by a “blinded” investigator using the modified Garcia scoring system (Shimamura et al. J Neurosci Methods, (2006), 156:161-165).
Infarct volume, hemispheric swelling, and excess water at 24 hours (Bertrand et al., J Vis Exp, (2017). Coronal brain sections were stained with 2,3,5-triphenyltetrazolium chloride (TTC). Images of TTC-stained sections were processed using the NIH ImageJ software 1.52a with an open-source, semi-automated plug-in, which features automatic-thresholding that yields reliable, unbiased measurements of TTC-negative and hemisphere areas (Friedlander et al., J Cereb Blood Flow Metab, (2017), 37:3015-3026). Preliminary infarct volume (mm³) was calculated based on the software-determined TTC-negative infarct area and slice thickness. To correct for tissue swelling, the preliminary infarct volume was divided by the swelling factor, calculated as ipsilateral hemisphere area/contralateral hemisphere area. Hemispheric swelling was calculated as (ipsilateral hemisphere volume/contralateral hemisphere volume)-1, expressed as percent. In separate experiments, excess water was measured using the wet weight/dry weight method.
Immunohistochemistry was performed as we described using primary antibodies directed against: SGLT2 (#NBPI-92384; Novus Biologicals, Centennial, CO), NeuN (#ABN90P; MilliporeSigma, Burlington, MA) and GFAP (#C9205, MilliporeSigma, Burlington, MA) (Tsymbalyuk et al., Mol Pain, (2021), 17:17448069211006603).
RNAScope was performed using a commercial kit (Multiplex Fluorescent Detection v2 kit, ACD, Newark, CA) according to the manufacturer's protocol.
Astrocyte isolation, qPCR and immunoblot were performed as we described, using (5′ to 3′: forward CAGACCTTCGTCATTCTTGCCG (SEQ ID NO:1); reverse GTGCTGGAGATGTTGCCAACAG (SEQ ID NO:2)) and a primary antibody directed against SGLT2 (cat #NBPI-92384; Novus) (Stokum et al., J Cereb Blood Flow Metab, (2021), 41:2546-2560).
Astrocyte cell volume. Mice with astrocyte-specific expression of tdTomato were obtained by crossing ROSA26-tdTomato mice (B6.Cg-Gt (ROSA) 26Sor^{tm14(CAG-tdTomato) Hze}/J; cat. #007914; Jackson Laboratories) with GFAP-Cre mice (B6.Cg-Tg (Gfap-cre) 73.12Mvs/J; cat. #012886; Jackson Laboratories). These mice were subjected to MCAo/R ( 2/6 hours). Coronal slices, 200 μm thick, from +1.0 mm to −2.0 mm relative to bregma were prepared (VT1200S, Leica) using ice-cold slicing aCSF bubbled with carbogen (95% 02/5% CO₂). Imaging was performed using a spinning disc confocal microscope (Nikon CSU-W1). Z-stack images of tdTomato-positive astrocytes were acquired at 5-minute intervals during a 30-minute protocol. Images were acquired in baseline aCSF containing 2 mM glucose for 2.5 minutes. SGLT2 was activated by switching to aCSF containing 10 mM glucose for the remainder of the experiment. Images were processed using the NIH ImageJ software to correct for x-y drift and fluorescence fading (Shigetomi et al., J Gen Physiol, (2013), 141:633-647). The areas of individual astrocytes for each Z-plane were used to determine cell volume.
Data analysis. Data are presented as mean+S.E. Student's t-test, 1-way ANOVA with Bonferroni post-hoc comparisons, or Mann-Whitney U test were used, as appropriate. The relationship between hemispheric swelling and infarct volume was analyzed using the equation (Eq. 1): Y=Y_{M} [1-exp (−k*X)], where X is infarct volume (mm³), Y is hemispheric swelling (%), Y_{M} is the maximum hemispheric swelling (%), and k is the rate of change (1/mm³). Analyses were performed with Origin Pro V8 or GraphPad Prism 8.3. P<0.05 was deemed to be statistically significant.

### Results

### SGLT2 Expression

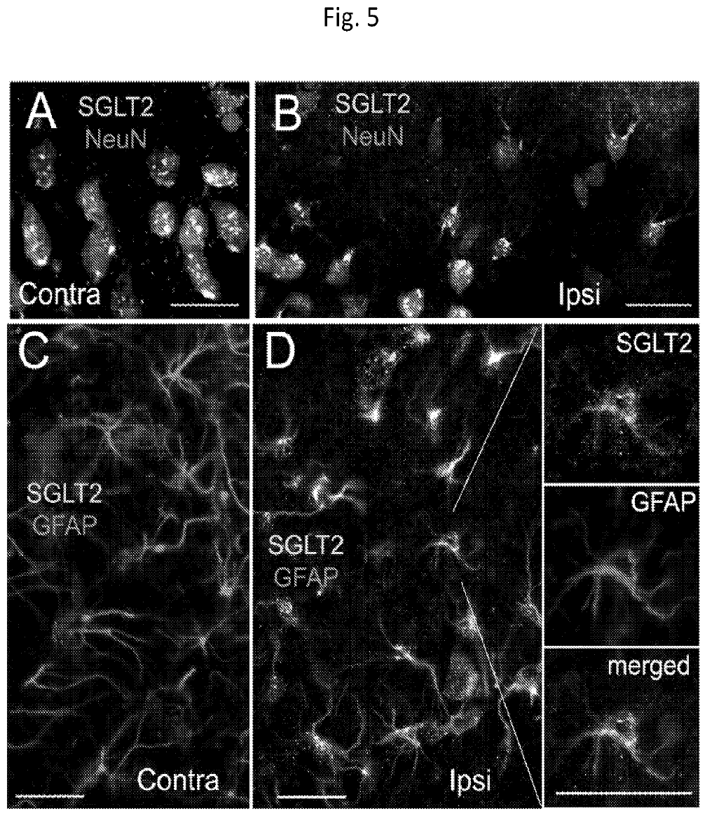

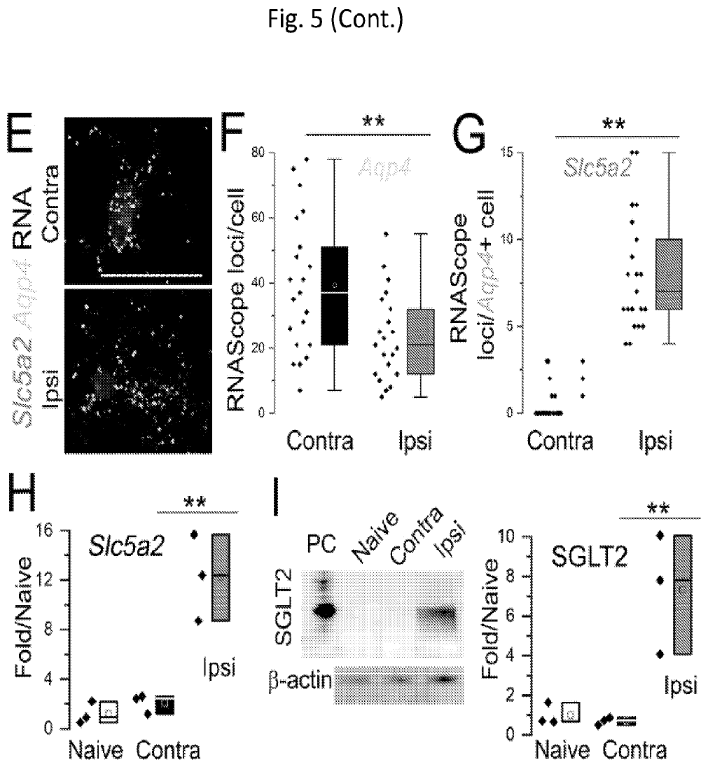

Immunolabeling for SGLT2 was performed on brain sections from mice following MCAo/R ( 2/24 hours). Sections were co-immunolabeled to identify cell-specific expression. SGLT2 was identified in NeuN-positive neurons, both ipsilateral and contralateral to MCAo/R, with no apparent difference due to ischemia (FIG. 5A-B). In GFAP-positive astrocytes, SGLT2 expression was minimal in contralateral tissues but was prominent ipsilateral to MCAo/R(FIG. 5C-D).

Astrocyte upregulation of SGLT2 was corroborated in tissue sections using RNAScope. Ipsilateral tissues showed slightly reduced mRNA for aquaporin-4 (Aqp4) (Stokum et al., Acta Neuropathol Commun, (2015), 3:61). However, mRNA for Slc5a2 was significantly increased in Aqp4-positive cells (FIG. 5E-G).

Further corroboration of astrocyte upregulation of SGLT2 was obtained by isolating astrocytes from mice following MCAo/R ( 2/6 hours). qPCR demonstrated ˜12-fold increase in Slc5a2 mRNA in ipsilateral vs. contralateral astrocytes, comparable to the fold-increase for Slc5a2 mRNA determined by RNAScope for Aqp4-expressing cells in tissue sections. Immunoblot showed minimal SGLT2 protein in contralateral astrocytes but prominent expressing in ipsilateral astrocytes (FIG. 5H-I).

### SGLT2 and Astrocyte Swelling In Vitro

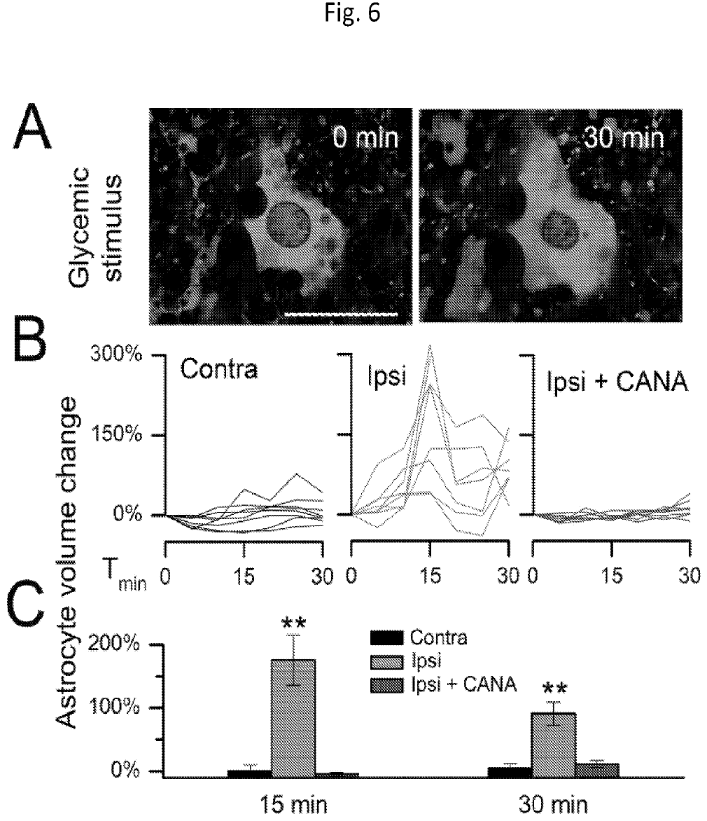

To study the function of newly upregulated SGLT2 in astrocytes, we performed MCAo/R ( 2/6 hours) in ROSA26-tdTomato;+GFAP-Cre mice, which express the fluorescent protein tdTomato in astrocytes. Ex vivo brain slices from these mice were used to image astrocytes, and SGLT2 was activated by exposing brain slices to a glycemic stimulus, which consisted of a step-change from 2 to 10 mM glucose. In slices from contralateral brain, astrocytes volumes were minimally affected by the glycemic challenge (FIG. 6). By contrast, in slices from ipsilateral brain, astrocytes exhibited a marked phasic increase in volume that subsided only partially, resulting in a persistent increase in volume that was significantly greater than in controls (FIG. 6A-C). Canagliflozin completely blocked the glucose-induced changes in astrocyte volume in the ipsilateral hemisphere (FIG. 6B-C), consistent with the dominant involvement of SGLT2 in the glucose-induced swelling response.

Apart from astrocyte cell swelling, the glycemic challenge also induced nuclear shrinkage in ipsilateral astrocytes (FIGS. 6A and D). Nuclear shrinkage accompanied by asymmetrical shape changes are typical of an osmotic perturbation (Finan et al., J Cell Biochem, (2010), 109:460-467).

### SGLT2 and Brain Swelling In Vivo

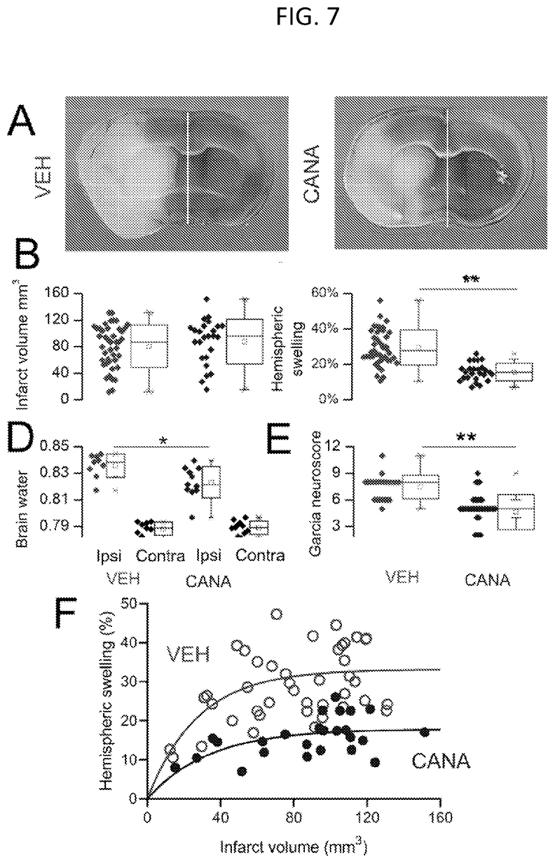

We studied the effect of canagliflozin on brain swelling in non-diabetic mice following MCAo/R ( 2/24 hours). Infarct volumes were not different in canagliflozin vs. vehicle groups (87.7±6.7 mm³ vs. 80.7±4.9 mm³; P=0.4), whereas hemispheric swelling was reduced by half in canagliflozin vs. vehicle groups, respectively (15.7±1.0% vs. 30.3±2.0%; P=l-7) (FIG. 7A-C). Blood glucose at reperfusion (175±10 mg/dL vs. 166±10 mg/dL; P=0.6; 5-7 mice/group) and at 24 hours (146±10 mg/dL vs. 139±14 mg/dL; P=0.7; 5-7 mice/group) was not different between groups.

The effect of drug on hemispheric swelling was corroborated in separate groups by measuring brain tissue water content. Ipsilateral brain water was reduced in canagliflozin vs. vehicle groups, respectively (82.3±0.4 vs. 83.6±0.3; P=0.016) (FIG. 7D).

Neurological function, measured using Garcia scores, was better in the canagliflozin vs. vehicle groups (medians: 8 vs. 5; P=41e-8) (FIG. 7E), consistent with the observed reduction in swelling.

The variability in infarct volume between subjects that is evident in FIG. 7B provided an opportunity to examine the relationship between infarct volume and hemispheric swelling. Individual data points are plotted in FIG. 7F. For infarct volumes less than ˜80 mm³, hemispheric swelling varied with infarct volume whereas with larger infarcts, swelling plateaued. The data were fit to a simple two-parameter exponential function, which showed that canagliflozin reduced swelling at all infarct volumes. The maximum swelling determined by fitting was 17.9% vs. 33.2% (P<0.0001) in canagliflozin-vs. vehicle-treated mice.

### DISCUSSION

The MCAo/R model in the C57BL6 mouse strain yields variable infarct volumes due to variability in collateral circulation, especially the posterior communicating artery (Knauss et al., Front Neurosci, (2020), 14:576741). Although typically viewed as a confounder in conventional preclinical stroke studies, we leveraged this variability to quantify the relationship between ipsilateral hemispheric swelling and infarct volume. Infarct volumes were not materially affected by canagliflozin. However, in both treatment groups, ipsilateral hemispheric swelling varied with infarct volume, consistent with brain swelling being dependent on infarct size (Kondo et al., J Neurosci, (1997), 17:4180-4189; Park et al., Acta Neurochir Suppl, 1997; 70:17-19). The data on swelling and infarct size from both groups were well fit to a simple exponential function. This analysis, based on large cohorts, demonstrated maximum swelling of 17.9% vs. 33.2% with canagliflozin vs. vehicle, respectively. The reduction in brain swelling with drug was mirrored by the reduction in excess brain water with drug. Overall, our data are consistent with canagliflozin reducing brain swelling independently of infarct volume.

The aim of stroke care in humans is first to minimize infarct size and maximize neurological recovery. Critical advances in thrombectomy over recent years have revolutionized the care of stroke patients. The next steps in improving stroke outcomes entail the development of effective agents to salvage neurons and other cells, and the development of effective strategies to minimize secondary injury, the most important in the acute phase being brain swelling. Reducing brain swelling is of great clinical significance, especially in LHI (Ng et al., Stroke, (2021), 52:3450-3458). Reducing brain swelling independently of infarct size was first demonstrated in a rodent model of MCAo using antisense oligodeoxynucleotides to target SUR1-TRPM4.7 In humans, the Phase 2 trial of BIIB093 targeting SUR1-TRPM4 provided strong support for the importance of reducing brain swelling in LHI (Sheth et al., Lancet Neurol, (2016), 15:1160-1169; Kimberly et al., Neurology, (2018), 91: e2163-e2169; Sheth et al., Stroke, (2018), 49:1457-1463; Vorasayan et al., Stroke, (2019), 50:3021-3027; Irvine et al., Cell Rep Med, (2022), 100654).

The data presented here suggest that canagliflozin targeting SGLT2 may be another promising agent in the quest to reduce brain swelling independently of infarct size. Canagliflozin is approved by the US Food and Drug Administration for patients with T2DM and in these patients it reduces blood glucose by inhibiting glucose reabsorption in the kidney. At the dose used here in non-diabetic mice, we did not find any important effect of canagliflozin on blood glucose. The absence of an effect on blood glucose in non-diabetic mice differs from observations with a related compound, empagliflozin, in a rat model with type 1 diabetes mellitus and 30-minute bilateral ischemia, in which empagliflozin reduced blood glucose as well as infarct volume (brain swelling was not studied) (Abdel-Latif R G et al., Arch Pharm Res, (2020), 43:514-525; Amin et al., Fundam Clin Pharmacol, (2020), 34:548-558). Since we found that the effect of canagliflozin on brain swelling was independent of blood glucose, and since the gliflozins readily penetrate the BBB, the effect that we observed on brain swelling likely was mediated via SGLT2 expressed by brain cells, especially astrocytes (Pawlos et al., Molecules, (2021), 26). The better neurological function with canagliflozin treatment in our model, despite no effect on infarct size, underscores the important benefit of reduced brain swelling on neurological function.

Apart from their demonstrated ability to lower blood glucose in T2DM patients, SGLT2 inhibitors also exert surprising protective effects. Regardless of the presence or absence of T2DM, long-term use of empagliflozin reduces the risk of death from cardiovascular disease and slows the progression of kidney disease (Seoudy et al., Dtsch Arztebl Int, (2021), 118; McMurray et al., N Engl J Med, (2019), 381:1995-2008; Packer et al., N Engl J Med, (2020), 383:1413-1424). These unexpected findings have fostered efforts to unravel the mechanisms linking SGLT2 inhibition and cardiorenal protection. The work presented here on cerebral ischemia extends the findings from the heart and kidney to the post-stroke brain. Our findings on SGLT2 involvement in glucose-induced cell swelling and nuclear shrinkage in astrocytes are novel, and have important implication not only for brain swelling but also for gene expression in the context of hyperglycemia (Finan et al., J Cell Biochem, (2010), 109:460-467).

In summary, ischemia sets in motion a program that culminates in cell death, especially neuronal death, that is marked by the loss of TTC processing by mitochondrial succinate dehydrogenase. As cell death proceeds, another program is initiated that is responsible for brain swelling. It has long been known that the two programs are closely linked but heretofore it has not been clear whether the two are separable. The data presented here with canagliflozin, as well as previous data on SUR1-TRPM4, indicate that the two programs are distinct and separable-that post-ischemic brain swelling is regulated by druggable cellular/molecular mechanisms that are distinct from those that govern neuronal death. Moreover, treatments that reduce brain swelling result in better neurological function even absent an effect on infarct size, underscoring the clinical importance of the finding that the two processes are distinct and independently treatable (Pawlos et al., Molecules, (2021), 26).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A method of treating reperfusion edema associated with ischemic stroke in a patient in need thereof, comprising administering to the patient an effective amount of one or more inhibitors of sodium D-glucose cotransporter 2 (SGLT2).

2. The method of claim 1, wherein the inhibitor is a member of the gliflozin family of drugs.

3. The method of claim 1, wherein the inhibitor is selected from the group consisting of canagliflozin, dapagliflozin, empagliflozin, ertugliflozin, ipragliflozin, luseogliflozin, remogliflozin etabonate, sergliflozin etabonate, sotagliflozin, tofogliflozin and combinations thereof.

4. The method of claim 1, wherein the SGLT2 inhibitor reduces reperfusion edema when administered after the patient has suffered an ischemic stroke.

5. The method of claim 1, wherein administration of the SGLT2 inhibitor reduces reperfusion edema in a manner that is independent of infarct volume.

6. The method of claim 1, wherein the SGLT2 inhibitor is administered in combination with an effective amount of one or more drugs or treatments for stroke.

7. The method of claim 6, wherein the SGLT2 inhibitor is administered in combination with an effective amount of recombinant tissue plasminogen activator (rtPA).

8. The method of claim 1, wherein the SGLT2 inhibitor is administered in combination with a mechanical thrombectomy procedure.

9. The method of claim 1, wherein administration of the SGLT2 inhibitor results in an increased time window for performing a thrombectomy procedure.

10. The method of claim 1, wherein administration of the SGLT2 inhibitor does not significantly affect serum glucose level of the patient.

11. A pharmaceutical composition comprising an effective amount of one or more inhibitors of sodium D-glucose cotransporter 2 (SGLT2) and an effective amount of one or more agents to treat ischemic stroke.

12. The composition of claim 11, wherein the composition comprises recombinant tissue plasminogen activator (rtPA).


SEQUENCE LISTING (2 sequences)

SEQ ID NO: 1 - DNA, 22 bp, synthetic construct
  source 1..22: mol_type = other DNA
cagaccttcgtcattcttgccg

SEQ ID NO: 2 - DNA, 22 bp, synthetic construct
  source 1..22: mol_type = other DNA
gtgctggagatgttgccaacag